(19) United States
(12) Patent Application Publication
(10) Pub. No.: US 2002/0137692 A1
(43) Pub. Date: Sep. 26, 2002

(54) Novel compositions of potassium channel openers and protein kinase C inhibitors and use thereof

(75) Inventors: Jan Rundegren, Loddekopinge (SE); Bo Kreilgaard, Hillerod (DK)

Correspondence Address: S. Maurice Valla, WOODCOCK WASHBURN LLP, One Liberty Place - 46th Floor, Philadelphia, PA, 19103, US

(73) Assignee: Pharmacia AB

(21) Appl. No.: 10/059,658

(22) Filed: Jan. 29, 2002

Related U.S. Application Data
(60) Provisional application No. 60/270,447, filed on Feb. 21, 2001.

(30) Foreign Application Priority Data
Feb. 7, 2001 (SE) SE 0100374-8

(51) Int. Cl. A61K 31/7048; A61K 31/685; A61K 31/506

(52) U.S. Cl. 514/27; 514/275; 514/422; 514/77; 514/550; 514/33

(57) ABSTRACT

Novel compositions comprising a potassium channel opener, such as minoxidil, and a protein kinase C inhibitor, such as procyanidin B2 are presented, as are kits containing a first composition comprising a potassium channel opener and a second composition comprising a protein kinase C inhibitor. Also presented are methods for using the novel compositions for treating and preventing hair loss in a region of a patient.

### CROSS REFERENCE TO RELATED APPLICATIONS

The present application also claims the benefit of priority under 35 U.S.C. § 119(e) from provisional U.S. Application Serial No. 60/270,447, filed on Feb. 21, 2001, which is incorporated herein by reference in its entirety.

The present application claims the benefit of priority under 35 U.S.C. § 119(a) from Swedish application SE 0100374-8, filed Feb. 7, 2001, which is incorporated herein by reference in its entirety.

### FIELD OF THE INVENTION

The present invention relates to novel compositions of potassium channel openers and protein kinase C inhibitors, methods for making the compositions, and methods for inducing and/or stimulating hair growth and/or reducing hair loss using the compositions. The present invention also relates to a first composition comprising one or more potassium channel openers and a second composition comprising one or more protein kinase C inhibitors, said first and second compositions to be used in combination for inducing and/or stimulating hair growth and/or reducing hair loss.

### BACKGROUND OF THE INVENTION AND PRIOR ART

Several potassium channel openers (PCOs), also called potassium channel agonists, have been shown to promote hair growth both in animals and humans with androgenetic alopecia. Examples of such PCOs are pinacidil, the pinacidil analogue P-1075 being N-cyano-N-tertpentyl-N′-3-pyridinylguanidine, diazoxide, cromakilin and minoxidil. See Buhl A E, Conrad S J, Waldon D J, Brunden M N. Potassium channel conductance as a control mechanism in hair follicles. J Invest Dermatol 1993; 101 (1 Suppl): pp. 148S-152S, and Harmon C S, Lutz D, Ducote J. Potassium channel openers stimulate DNA synthesis in mouse epidermal keratinocyte and whole hair follicle cultures. Skin Pharmacol 1993; 6: 170-178. These PCOs have also vascular effects in that they relax smooth muscle cells of capillaries. This property has been implicated as a possible mechanism for stimulating hair growth by increasing blood supply to the hair follicles, but other mechanisms as well have been suggested for the hair growth stimulant minoxidil.

Such mechanisms are anti-fibrotic effect by its inhibition of lysyl hydroxylase (Murad S, Pinell S R. Suppression of fibroblast proliferation and lysyl hydroxylase activity by minoxidil. J. Biol Chem 1987; 262:11973-78), stimulation of keratinocytes (Harmon C S, Lutz D, Ducote J. Potassium channel openers stimulate DNA synthesis in mouse epidermal keratinocyte and whole hair follicle cultures. Skin Pharmacol 1993; 6: 170-178, and Boyera N, Galey I, Bernard B A. Biphasic effects of minoxidil on the proliferation and differentiation of normal human keratinocytes. Skin Pharmacol 1997; 10: 206-220), stimulation of various growth factors (Lachgar S, Charveron M, Gall Y, Bonafe J L. Minoxidil upregulates the expression of vascular endothelial growth factor in human hair dermal papilla cells. Br J Dermatol 1998; 138: 407-411, and Yamazaki M, Tsuboi R, Lee Y R, Ishido K, Mitsui S, Ogawa H. Hair cycle-dependent expression of hepatocyte growth factor (HGF) activator, other proteinases, and proteinase inhibitors correlates with the expression of HGF in rat hair follicles. J Invest Dermatol Symp Proc 1999; 4: 312-315), reduced collagen synthesis (Lachgar S, Charvéron M, Bouhaddioui, Eveux Y, Gall Y, Bonafé. Inhibitory effects of bFGF, VEGF and minoxidil on collagen synthesis by cultured hair dermal papilla cells. Arch Dermatol Res 1996; 288: 469-473) and activation of the cytoprotective prostaglandin PGHS-1 (Michelet J F, Commo S, Billoni N, Mahe Y F, Bernard B A. Activation of cytoprotective prostaglandin synthase-1 by minoxidil as a possible explanation for its hair growth-stimulating effect. J Invest Dermatol 1997; 108: 205-209).

Minoxidil (being 2,4-diamino-6-piperidinylpyrimidine-3-oxide) is the active ingredient of Loniten® and Rogaine®, which are marketed by Pharmacia as a treatment for hypertension, and as a treatment and preventative for androgenic alopecia (male and female pattern baldness), respectively. The preparation and antihypertensive use of minoxidil is described in U.S. Pat. No. 3,461,461. Methods and topical preparations for using the compound to grow hair and to treat male and female pattern baldness are described and claimed in U.S. Pat. Nos. 4,139,619 and 4,596,812.

It has recently been shown that several selective protein kinase C inhibitors promote hair growth. See Takahashi T, Kamimura A, Shirai A, Yokoo Y. Several selective protein kinase C inhibitors including procyanidins promote hair growth. Skin Pharmacol Appl Skin Physiol 2000; 13: 133-142. Among those are found calphostin C, procyanidin C1, hexadecylphosphocholine, palmitoyl-DL-carnitine chloride, polymyxin B sulfate, and procyanidin B2. Those molecules have all showed marked anagen induction in vivo and have not been associated with potassium channel agonism. When phenolic compounds extracted from plants were investigated for possible hair growth it was found that proanthocyanidins were capable to induce anagen phase. Proanthocyanidins are polymers or oligomers of flavan-3-ol units, such as catechin, epicatechin, gallocatechin, epigallocatechin, afizelechin, and epiafzelechin; whose molecules occasionally incorporate gallic acid. See Porter L J. Flavans and proanthocyanidins. In The Flavonoids: Advances in Research Since 1986, ed Harborne J B, pp. 23-55. Chapman and Hall, London 1994, and Takahashi T, Kamimura A, Shirai A, Yokoo Y. Several selective protein kinase C inhibitors including procyanidins promote hair growth. Skin Pharmacol Appl Skin Physiol 2000; 13: 133-142.

Epicatechin dimers such as procyanidin B1, B2, and B3 as well as the epicatechin trimer C1 but not the monomer show stimulation of hair keratinocytes and hair growth stimulation in the C3H mouse. See Takahashi T, Kamiya T, Hasegawa A, Yokoo Y. Procyanidin oligomers selectively and intensively promote proliferation of mouse hair epithelial cells in vitro and activate hair follicle growth in vivo. J Invest Dermatol 1999; 112: 310-316.

Use of proanthocyanidine as hair growth promoter is disclosed in WO 9600561.

There are disclosed combination products comprising a potassium channel opener and some other hair promoting agent, e g minoxidil and the 5-alpha reductase inhibitor finasteride, U.S. Pat. No. 5,578,599, and minoxidil and the androgen receptor RU 58841, U.S. Pat. No. 5,411,981.

Anyhow, nowhere is disclosed to combine one or more potassium channel openers and one or more protein kinase C inhibitors.

By combining in a formulation one or more potassium channel openers and one or more protein kinase C inhibitors, which are characterized by different mechanisms of action, an increased hair growth stimulation is achieved in comparison to having just one of the two compound types in the formulation. The two compound types can be mixed and combined in a topical product for treatment of hair loss. The present invention is directed to these, as well as other, important ends. The disclosures of each patent, patent application and publication cited or described in this document are hereby incorporated herein by reference, in their entirety.

### SUMMARY OF THE INVENTION

The present invention is directed to first novel compositions of one or more potassium channel openers and one or more protein kinase C inhibitors. Specifically, in one embodiment, there is provided a composition comprising minoxidil and procyanidin B2.

Other embodiments of the invention relate to first compositions comprising combinations of other potassium channel openers and other protein kinase C inhibitors.

Further embodiments of the invention relate to a second composition comprising one or more potassium channel openers and a third composition comprising one or more protein kinase C inhibitors, said first and second compositions to be used in combination for inducing and/or stimulating hair growth and/or reducing hair loss.

Methods for treating and/or preventing hair loss in a region of a patient, wherein the methods comprise topically administering to the region compositions as described herein, are also provided by the present invention.

These and other aspects of the invention will become more apparent from the present disclosure and claims.

### DETAILED DESCRIPTION OF THE INVENTION

The present invention is directed, in part, to novel first compositions of one or more potassium channel openers and one or more protein kinase C inhibitors, to methods for making the compositions, and to methods for inducing and/or stimulating hair growth and/or reducing hair loss using the compositions. The present invention also relates to a second composition comprising one or more potassium channel openers and a third composition comprising one or more protein kinase C inhibitors, said second and third compositions to be used in combination for inducing and/or stimulating hair growth and/or reducing hair loss.

As noted above, the preparation and antihypertensive use of minoxidil is described in U.S. Pat. No. 3,461,461, and topical preparations and methods relating to the use of the compound to grow hair and to treat androgenic alopecia are described and claimed in U.S. Pat. Nos. 4,139,619 and 4,596,812. The disclosures of these three patents are hereby incorporated herein by reference, in their entireties.

As noted above the use of protein kinase C inhibitors for treating alopecia is disclosed in captioned Takahashi et al 1999 and 2000.

The compositions of the present invention may take a variety of forms including, for example, solutions, emulsions, suspensions or mixture thereof in a wide range of viscosities, including semi-solids, in gelled or non-gelled form, for direct topical application or for topical application by spraying, and different forms of patches for topical application.

The respective concentration of the one or more potassium channel openers and the one or more protein kinase C inhibitors in the present compositions may vary within a wide range depending on the specificity for the respective compound and the pharmaceutical formulation used. Useful concentrations are though from around 0.5% (w/w) to around 10% (w/w) when the potassium channel opener minoxidil and from around 0.1% (w/w) to around 10% (w/w) when the protein kinase C inhibitor is procyanidin B2.

A wide variety of solvents may be used in the compositions of the present invention. Preferably, the solvent is a polar solvent. Preferred among these are polar, protic solvents. Preferably, the solvent is water or a hydroxy compound, ie, a compound containing at least one hydroxy (OH) group. Preferred among the hydroxy compounds are alcohols (ie, compounds containing one hydroxy group) or polyols (ie, compounds containing two or more hydroxy groups) or mixtures of alcohols and/or polyols. Exemplary alcohols include, for example, ethanol, propanol and butanol. Reference herein to “ethanol” includes absolute alcohol, as well as “alcohol USP” and all denatured forms of 95% ethanol. As used herein, the term “propanol” refers to all isomeric forms, including n-propanol and isopropanol, and the term “butanol” refers to all isomeric forms, including, for example, n-butanol, iso-butanol and sec-butanol. Preferred among these alcohols are ethanol and propanol, with ethanol being more preferred. Exemplary polyols include, for example propylene glycol, dipropylene glycol, hexylene glycol, 1,3-butylene glycol, liquid polyethylene glycols, such as polyethylene glycol 200 (PEG-200) and polyethylene glycol 400 (PEG-400), and glycerol (the latter also referred to sometimes as glycerine). Preferred among these polyols is propylene glycol. In a particularly preferred embodiment, the solvent employed may be a mixture of water, an alcohol and a polyol.

The compositions of the present invention may be topically administered to a region of a patient for the prevention or treatment of hair loss. Accordingly, as would be apparent to one of skill in the art, once armed with the teachings of the present disclosure, the compositions may optionally comprise additional pharmaceutically acceptable additives and ingredients such as, for example, pH modifiers, chemical stabilizers, including antioxidants, hair conditioners, such as vitamin B5/panthenol, calcium pantothenate or other panthenol derivatives, colorants, fragrances, fragrance modifiers, other vitamins such as vitamin E, penetration modifiers, such as azone and DEET, surfactants, such as Cremophor® (BASF), cosmetic agents for the skin or scalp, such as fatty acids and fatty acid esters, herbal extracts, such as henna, other viscosity enhancing or thickening agents, oils, emulsifiers, wetting agents, sunscreens and anti-irritants.

A wide variety of methods may be used for preparing the compositions of the present invention. Broadly speaking, the compositions may be prepared by combining together the components of the compositions, as described herein, at a temperature and for a time sufficient to preferably provide a pharmaceutically elegant composition. The term “combining together”, as used herein, means that all of the components of the compositions may be combined and mixed together at about the same time. In certain preferred embodiments, the term “combining together” means that the various components may be combined in one or more preferential sequences to provide the desired product.

The compositions of the present invention may be advantageously employed to treat and/or prevent a region of hair loss or alopecia in a patient. Generally speaking, the methods may comprise topically administering to the region a composition as described herein. The life of a hair is subjected to a cycle, called the pilar cycle, during which the hair grows (anagen), transitions (catagen), and falls out (telogen), before being replaced by a new hair which appears in the same follicle and the cycle is repeated. This constant renewal process undergoes a natural change during ageing. The hair cycles become shorter, resulting in finer, shorter hairs. Hair loss results when this process is accelerated or disturbed, i.e. the growth phases become shorter, the passage of hair into the telogen phase is earlier and hairs fall out in larger numbers. Successive shortening growth cycles may result in increasingly fine and short hair, which is slowly converted into fluff. This phenomenon may lead to progressive hair thinning and may eventually lead to baldness.

Dermatologists recognize many different types of hair loss, the most common by far being androgenetic alopecia (also known as male or female “pattern baldness”), wherein humans begin losing scalp hair as they get older. While this type of hair loss is more common in males, it also occurs in women. This male type of alopecia may be characterized by progressive thinning, as discussed above, or may be characterized by hair loss with little diffuse hair thinning, such as frontal hair loss, mid-anterior balding, bitemporal recession, and/or vertex balding. In females, the androgenetic alopecia is generally characterized by a diffuse thinning of the top of the scalp but with preservation of a frontal hairline. Alopecia areata, anagen hair loss, and diffuse alopecia, such as telogen effluvium are other presentations of hair loss, which may be distinguished from androgenetic alopecia. These other forms of hair loss may also be treated with the present compositions.

The invention is further described in the following examples. These examples are for illustrative purposes only, and are not to be construed as limiting the appended claims.

In the absence of explicit statements to the contrary, as used herein expressions like “comprising”, “including”, “having”, “with” and similar terminology shall not be understood to be exclusively restricted to recited element, but shall be understood to allow for the presence of further elements as well, and shall be understood to cover any element in integral, sub-divided or aggregate forms, as well to imply the inclusion of a stated integer or step or group of integers or steps, but not the exclusion of any other integer or step or group of integers or steps.

As compositions according to the present invention have a better hair growing effect than compositions comprising either only potassium channel openers or only protein kinase C inhibitors it is possible, in order to achieve the same effect, to administer the present compositions less frequently than had instead been administered a composition comprising either only potassium channel openers or only protein kinase C inhibitors. Useful administration of the present compositions hence includes administration once daily or even less often.

### EXAMPLES

### Example 1

Preparation of Solution 1

500 mg minoxidil, 200 mg procyanidin B2, 5.0 g propyleneglycol, 3.0 ml ethanol and purified water to make 10 ml was mixed at room temperature. The mixture was stirred until a clear reddish-brown solution was obtained.

### Example 2

Preparation of Solution 2

A batch of 100 ml was obtained by mixing 0.3% (w/w) procyanidin B2, 2.0% (w/w) minoxidil, 70% (w/w) ethanol, 10% (w/w) 1,3-butylene glycol and 18% (w/w) purified water was mixed and stirred at room temperature to obtain a clear reddish-brown solution.

### Example 3

Preparation of Alternative Solutions

As alternatives to Solution 1 and Solution 2 are manufactured solutions essentially in accordance with Examples 1 and 2 wherein the solvent(s) instead are chosen from the list of suitable solvents on page 4.

### Example 4

Preparation of Alternative Embodiments

As alternatives to solutions according to Examples 1, 2 or 3 may be manufactured a second composition comprising one or more potassium channel openers and a third composition comprising one or more protein kinase C inhibitors, said first and second compositions to be used in combination for inducing and/or stimulating hair growth and/or reducing hair loss. Said second and third compositions are manufactured essentially according to Examples 1, 2 or 3.

Said second and third compositions may be administered in many different dosage regimes, eg concomitantly or irrespective of one another. For example a patient may administer one dose from each composition in the morning and one dose from each composition in the evening. Alternatively a patient may administer one dose from the second composition in the morning and one dose from the third composition in the evening. Also other regimes are envisageable, such as different number of doses per day for the second and the third composition respectively. The most suitable administration regime depends on the factual therapeutic situation.

### Example 5

Analysis of Minoxidil

The quantitative determination of minoxidil was performed by means of a HPLC method using a column (Symmetry™), C18, 3.5 μm, 100 Å, 150×4.6 mm and a water/methanol/glacial acid (30/70/1) mobile phase with 3 g/l of docusate sodium and adjusted to pH 3.0 using perchloric acetic acid. The flow was 1.0 ml/min. the injection volume 20 μl and detection was done at 254 nm using an UV-detector.

1.0 ml of sample was diluted to 100 ml with ethanol and an aliquot of this mixture was diluted 1:50 with mobile phase. A standard solution contained 10 μ/ml of minoxidil. Sample and standard solution were injected and the content of minoxidil calculated based on peak response.

### Example 6

Analysis of Procyanidin B2

Qualification of procyanidin B2 was performed by means of a HPLC method using a column (Symmetry™), C18, 3.5 μm, 100 Å, 150×4.6 mm and a water/acetonitrile/glacial acid (93:5.2) mobile phase. The flow was 1.0 ml/min. the injection volume 10 μl and detection was done at 280 nm using an UV-detector.

0.5 ml of sample was diluted to 25 ml with mobile phase. A standard solution contained 50 μ/ml of procyanidin B2. Sample and standard solution were injected and the content of procyanidin B2 calculated based on peak response.

### Example 7

Stability of Solution 1 and Solution 2 is disclosed below in Table 1 and Table 2 respectively.

TABLE 1
Solution 1

| | | | Procyanidin B2, |
|---|---|---|---|
| Time | Temperature | Minoxidil, mg/ml | mg/ml |
| Initial | | 50.2 | 10.0 |
| 1 week | 8° C. | 49.1 | 9.5 |
| 2 weeks | 8° C. | | 9.7 |
| 6 weeks | 8° C. | 51.1 | |
| 8 weeks | 8° C. | | 10.0 |
| 1 week | 25° C. | 49.1 | 9.6 |
| 2 weeks | 25° C. | | 9.5 |
| 6 weeks | 25° C. | 51.0 | |
| 8 weeks | 25° C. | | 9.1 |

TABLE 2
Solution 2

| Time | Temperature | Minoxidil, mg/ml |
|---|---|---|
| 4 weeks | 5° C. | 19.4 |
| 4 weeks | 25° C. | 19.3 |
| 4 weeks | 40° C. | 19.7 |

The above results show that stable products are obtained.

### Monkey Study

The stumptail macaque monkey is used for testing effect of the substances on hair growth. This species show general androgenetic alopecia of the frontal area of the scalp already from the age of two years and has been shown to respond with hair growth to both topical minoxidil and oral finasteride. In contrast to mice stumptail macaque monkeys are the most universal model animals in this therapeutic area responding both to hormonal and non-hormonal hair growth promoting substances.

A one square inch test area is marked by tattooing dots at the corners of the square. At baseline the test area is shaved and after each month for 4 months the test area is shaved and the weight of the hair is measured. The hair weight is a measure of hair growth. The monkeys are studied in three groups with at least 5 animals in each group. The products tested in the three arms are a) 5% topical minoxidil, b) 1% topical procyanidin B2, c) a mixture of 5% minoxidil and 1% procyanidin B2 manufactured in the same way as Solution 1 of Example 1.

Various modification of the invention, in addition to those described herein, will be apparent to those skilled in the art from the foregoing description. Such modifications are also intended to fall within the scope of the appended claims.

The invention claimed is:

1. A composition comprising a potassium channel opener and a protein kinase C inhibitor.

2. A composition according to claim 1 wherein said potassium channel opener is selected from the group consisting of pinacidil, diazoxide, cromakilin, minoxidil, and analogs, salts, and derivatives thereof.

3. A composition according to claim 2 wherein said potassium channel opener is selected from the group consisting of minoxidil, and analogs, salts, and derivatives thereof.

4. A composition according to claim 3 wherein the concentration of said potassium channel opener is from about 0.5% (w/w) to about 10% (w/w).

5. A composition according to claim 1 wherein said protein kinase C inhibitor is selected from the group consisting of calphostin C, hexadecylphosphocholine, palmitoyl-DL-carnitine chloride, polymyxin B sulfate, proanthocyanidins, and analogs, salts, and derivatives thereof.

6. A composition according to claim 5 wherein said protein kinase C inhibitor is selected from the group consisting of procyandin B2, and analogs, salts, and derivatives thereof.

7. A composition according to claim 6 wherein the concentration of said protein kinase C inhibitor is from about 0.1% (w/w) to about 10% (w/w).

8. A composition according to claim 1 wherein said potassium channel opener is selected from the group consisting of pinacidil, diazoxide, cromakilin, minoxidil, and analogs, salts, and derivatives thereof, and said protein kinase C inhibitor is selected from the group consisting of calphostin C, hexadecylphosphocholine, palmitoyl-DL-carnitine chloride, polymyxin B sulfate, proanthocyanidins, and analogs, salts, and derivatives thereof.

9. A composition according to claim 8 wherein said potassium channel opener is selected from the group consisting of minoxidil, and analogs, salts, and derivatives thereof, and said protein kinase C inhibitor is selected from the group consisting of procyandin B2, and analogs, salts, and derivatives thereof.

10. A composition according to claim 8 further comprising a solvent selected from the group consisting of water and hydroxy compounds.

11. A composition according to claim 10 wherein said solvent is selected from the group consisting of water, ethanol, propanol, butanol, propylene glycol, dipropylene glycol, hexylene glycol, 1,3-butylene glycol, liquid polyethylene glycols and glycerol.

12. A composition according to claim 10 wherein said potassium channel opener and said protein kinase C inhibitor are dissolved in a mixture comprising propylene glycol, ethanol, and water.

13. A composition according to claim 10 wherein said potassium channel opener and said protein kinase C inhibitor are dissolved in a mixture comprising 1,3-butylene glycol, ethanol, and water.

14. A composition according to claim 10 wherein said potassium channel opener and said protein kinase C inhibitor are dissolved in a mixture comprising dipropylene glycol, ethanol, and water.

15. A composition according to claim 10 further comprising pharmaceutically acceptable ingredients selected from the group consisting of pH modifiers, chemical stabilizers, hair conditioners, panthenol derivatives, calcium pantothenate, colorants, fragrances, fragrance modifiers, vitamin E, penetration modifiers, surfactants, cosmetic agents, fatty acids, fatty acid esters, herbal extracts, henna, oils, emulsifiers, wetting agents, sunscreens, and anti-irritants.

16. A composition according to claim 10 formulated in a dosage form selected from the group consisting of solutions, emulsions, suspensions, and mixtures thereof, in gelled or non-gelled form.

17. A composition according to claim 16 formulated in a dosage form suitable for topical application by a method selected from the group consisting of direct application, application by spraying, and application by topical patch.

18. A kit comprising a first composition comprising a potassium channel opener and a second composition comprising a protein kinase C inhibitor.

19. A kit according to claim 18 wherein said potassium channel opener is selected from the group consisting of pinacidil, diazoxide, cromakilin, minoxidil, and analogs, salts, and derivatives thereof.

20. A kit according to claim 19 wherein said potassium channel opener is selected from the group consisting of minoxidil, and analogs, salts, and derivatives thereof.

21. A kit according to claim 20 wherein the concentration of said potassium channel opener in said first composition is from about 0.5% (w/w) to about 10% (w/w).

22. A kit according to claim 18 wherein said protein kinase C inhibitor is selected from the group consisting of calphostin C, hexadecylphosphocholine, palmitoyl-DL-carnitine chloride, polymyxin B sulfate, proanthocyanidins, and analogs, salts, and derivatives thereof.

23. A kit according to claim 22 wherein said protein kinase C inhibitor is selected from the group consisting of procyandin B2, and analogs, salts, and derivatives thereof.

24. A kit according to claim 23 wherein the concentration of said protein kinase C inhibitor in said second composition is from about 0.1% (w/w) to about 10% (w/w).

25. A kit according to claim 18 wherein said potassium channel opener is selected from the group consisting of pinacidil, diazoxide, cromakilin, minoxidil, and analogs, salts, and derivatives thereof, and said protein kinase C inhibitor is selected from the group consisting of calphostin C, hexadecylphosphocholine, palmitoyl-DL-carnitine chloride, polymyxin B sulfate, proanthocyanidins, and analogs, salts, and derivatives thereof.

26. A kit according to claim 25 wherein said potassium channel opener is selected from the group consisting of minoxidil, and analogs, salts, and derivatives thereof, and said protein kinase C inhibitor is selected from the group consisting of procyandin B2, and analogs, salts, and derivatives thereof.

27. A kit according to claim 25 wherein each of said first composition and said second composition further comprise a solvent selected from the group consisting of water and hydroxy compounds.

28. A kit according to claim 27 wherein each said solvent is selected from the group consisting of water, ethanol, propanol, butanol, propylene glycol, dipropylene glycol, hexylene glycol, 1,3-butylene glycol, liquid polyethylene glycols and glycerol.

29. A kit according to claim 27 wherein said potassium channel opener and said protein kinase C inhibitor are each dissolved in a mixture comprising propylene glycol, ethanol, and water.

30. A kit according to claim 27 wherein said potassium channel opener and said protein kinase C inhibitor are each dissolved in a mixture comprising 1,3-butylene glycol, ethanol, and water.

31. A kit according to claim 27 wherein said potassium channel opener and said protein kinase C inhibitor are each dissolved in a mixture comprising dipropylene glycol, ethanol, and water.

32. A kit according to claim 27 wherein said first composition and said second composition each further comprise pharmaceutically acceptable ingredients selected from the group consisting of pH modifiers, chemical stabilizers, hair conditioners, panthenol derivatives, calcium pantothenate, colorants, fragrances, fragrance modifiers, vitamin E, penetration modifiers, surfactants, cosmetic agents, fatty acids, fatty acid esters, herbal extracts, henna, oils, emulsifiers, wetting agents, sunscreens, and anti-irritants.

33. A kit according to claim 27 wherein each composition is formulated in a dosage form selected from the group consisting of solutions, emulsions, suspensions, and mixtures thereof, in gelled or non-gelled form.

34. A kit according to claim 33 wherein each composition is formulated in a dosage form suitable for topical application by a method selected from the group consisting of direct application, application by spraying, and application by topical patch.

35. A method for treating or preventing male or female hair loss in a region of a patient, wherein said method comprises topically administering to said region a composition according to any one of claims 1 to 17.

36. A method according to claim 35 wherein said hair loss is caused by a condition selected from the group consisting of androgenetic alopecia, alopecia areata, and diffuse alopecia.

37. A method according to claim 35 wherein said composition is administered once-a-day or less frequently than once-a-day.

38. A method for treating or preventing male or female hair loss in a region of a patient, wherein said method comprises topically administering to said region a first composition comprising a potassium channel opener and a second composition comprising a protein kinase C inhibitor.

39. A method according to claim 38, wherein said potassium channel opener is selected from the group consisting of pinacidil, diazoxide, cromakilin, minoxidil, and analogs, salts, and derivatives thereof, and said protein kinase C inhibitor is selected from the group consisting of calphostin C, hexadecylphosphocholine, palmitoyl-DL-carnitine chloride, polymyxin B sulfate, proanthocyanidins, and analogs, salts, and derivatives thereof.

40. A method according to claim 39, wherein said potassium channel opener is selected from the group consisting of minoxidil, and analogs, salts, and derivatives thereof, and said protein kinase C inhibitor is selected from the group consisting of procyandin B2, and analogs, salts, and derivatives thereof.

41. A method according to claim 38 wherein said first composition and said second composition are administered concomitantly.

42. A method according to claim 38 wherein said first composition and said second composition are administered independently of each other.

43. A method according to claim 38 wherein said hair loss is caused by a condition selected from the group consisting of androgenetic alopecia, alopecia areata, and diffuse alopecia.

44. A method according to claim 38 wherein each of said first and second compositions is administered once-a-day or less frequently than once-a-day.
